(19) United States
(12) Patent Application Publication
(10) Pub. No.: US 2007/0173490 A1
(43) Pub. Date: Jul. 26, 2007

(54) Novel solid forms of (4R)-1-[2-chloro-5-fluorobenzoyl)amino-3-methoxybenzoyl]-1,2,3,5- tetrahydro-spiro[4h-1-benzazepine-4,1'-[2]cyclopentene]-3'-carboxylic acid

(76) Inventors: Lian Huang, Basking Ridge, NJ (US); Wenju Wu, Piscataway, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/624,292

(22) Filed: Jan. 18, 2007

Related U.S. Application Data
(60) Provisional application No. 60/760,427, filed on Jan. 20, 2006.
(60) Provisional application No. 60/823,784, filed on Aug. 29, 2006.

(51) Int. Cl. A61K 31/55; C07D 223/14

(52) U.S. Cl. 514/212.02; 540/543

(57) ABSTRACT

The present invention relates to novel solid forms of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid (formula (I)) useful for treating and/or preventing conditions such as diabetic nephropathy, renal disease, renal failure and congestive heart failure.

### CROSS REFERENCE TO RELATED APPLICATION

This application claims the benefit of U.S. Provisional Application No. 60/760,427, filed Jan. 20, 2006 and U.S. Provisional Application No. 60/823,784, filed Aug. 29, 2006, which are incorporated by reference herein.

### FIELD OF THE INVENTION

The present invention relates to novel crystalline and non-crystalline forms of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, pharmaceutical compositions comprising such crystalline and non-crystalline forms, and methods of making and using the same.

### BACKGROUND OF THE INVENTION

Drugs in pharmaceutical compositions can be prepared in a variety of different forms. Such drugs can be prepared so as to have a variety of different chemical forms including chemical derivatives or salts. Such drugs can also be prepared to have different physical forms. For example, the drugs may be amorphous or may have different crystalline polymorphs. In addition, the existence of different solvation or hydration states are possible. By varying the form of a drug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility.

Chen et al., in PCT publication WO02/02531, disclose a process for the preparation of nonpeptide substituted spirobenzazepines. One such substituted spirobenzazepine is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, represented by the structure (I):

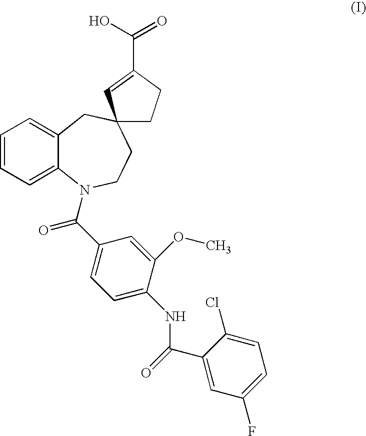

### SUMMARY OF THE INVENTION

The present invention relates to novel crystalline forms of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid (formula (I) below),

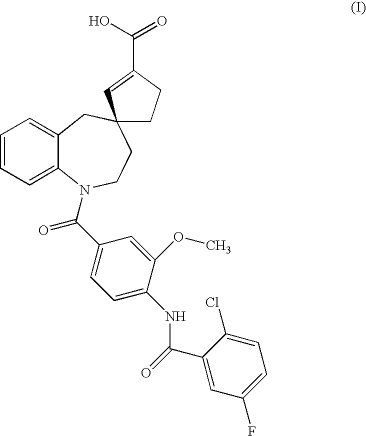

including polymorphs, hydrates, solvates, and amorphous forms. The invention also provides novel pharmaceutical compositions comprising one or more forms of the compound of formula (I), methods of making forms of the compound of formula (I), and related methods of treatment.

Compositions and methods of the invention are useful in the treatment or prevention of inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries.

Accordingly, in a first aspect, the present invention provides the following crystal forms of compound of formula (I):

a crystalline polymorph (form 1) of the compound of formula (I);
a crystalline toluene solvate (form 2) of the compound of formula (I);

a crystalline dichloromethane solvate (form 3) of the compound of formula (I);

a crystalline methanol solvate (form 4) of the compound of formula (I);

a crystalline polymorph (form 5) of the compound of formula (I);

a crystalline polymorph (form 6) of the compound of formula (I);

a crystalline acetonitrile solvate (form 7) of the compound of formula (I);

a crystalline ethyl acetate solvate (form 8) of the compound of formula (I);

a crystalline nitromethane solvate (form 9) of the compound of formula (I); and

an amorphous form (form 10) of the compound of formula (I).

For a better understanding of the present invention, together with other and further objects thereof, reference is made to the accompanying drawings and detailed description and its scope will be pointed out in the appended claims.

### BRIEF DESCRIPTION OF THE DRAWINGS

FIG. 1—PXRD diffractogram of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1

FIG. 2—PXRD diffractogram of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2

FIG. 3—PXRD diffractogram of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3

FIG. 4—PXRD diffractogram of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4

FIG. 5—PXRD diffractogram of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5

FIG. 6—PXRD diffractogram of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6

FIG. 7—PXRD diffractogram of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7

FIG. 8—PXRD diffractogram of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8

FIG. 9—PXRD diffractogram of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9

FIG. 10—PXRD diffractogram of amorphous (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid (form 10)

### DETAILED DESCRIPTION OF THE INVENTION

The present invention is directed to novel crystalline and amorphous forms of a nonpeptide substituted spirobenzazepine derivative useful for treating and/or preventing conditions such as increased vascular resistance and cardiac insufficiency. The novel crystalline forms include polymorphs and solvates of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid.

Patel et al. describe in US20040266752 A1 substituted spirobenzazepines of formula (II) having substituents as described therein,

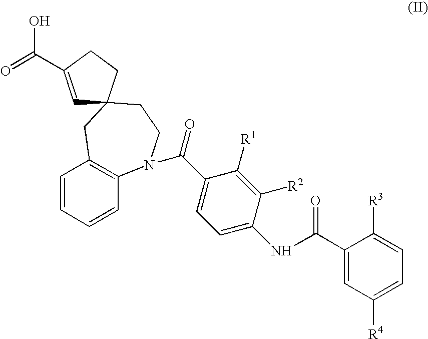

which includes the compound of formula (I):

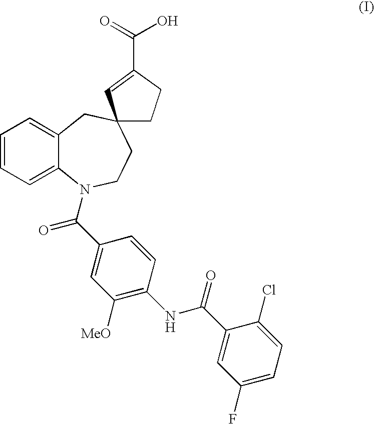

Patel et al. also describe methods of treating a subject suffering from, and inhibiting in a subject the onset or progression of, a condition associated with vasopressin receptor activity, which comprises administering to the subject a therapeutically or prophylactically effective amount of the compound of formula (II). In particular, such conditions includes inner ear disorders, hypertension, congestive heart failure, cardiac insufficiency, coronary vasospasm, cardiac ischemia, liver cirrhosis, renal vasospasm, renal failure, diabetic nephropathy, hyponatremia, cerebral edema, cerebral ischemia, stroke, thrombosis, water retention, aggression, obsessive-compulsive disorders, dysmenorrhea, nephrotic syndrome, anxiety and central nervous injuries.

In U.S. Pub. No. US20040259857A1, Deng et al. disclose an improved process for the preparation of nonpeptide substituted spirobenzazepine derivatives and novel processes for the preparation of intermediates in the preparation of said derivatives including the compound of formula (I). In particular, said compound of formula (I), (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, is a white solid as a free acid, which can be prepared according to, for example, the process outlined in Examples 1-4 of the instant disclosure.

In a first embodiment, the present invention comprises polymorphs of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid.

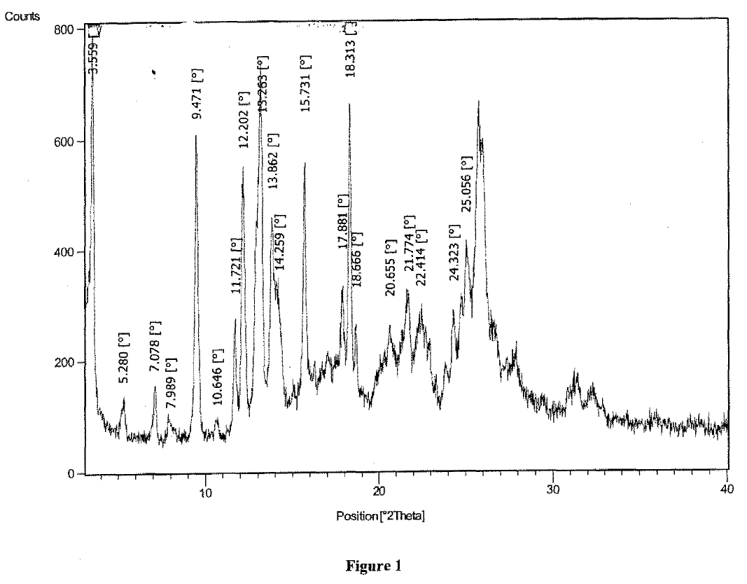

In a further embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by a PXRD diffractogram peak at about 9.47 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by a PXRD diffractogram peak at about 13.26 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by a PXRD diffractogram peak at about 22.41 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by PXRD diffractogram peaks at about 9.47 and about 13.26 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by PXRD diffractogram peaks at about 9.47 and about 12.20 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by PXRD diffractogram peaks at about 9.47 and about 20.66 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by PXRD diffractogram peaks at about 9.47, about 13.26, and about 15.73 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by PXRD diffractogram peaks at about 9.47, about 13.26, and about 22.41 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by PXRD diffractogram peaks at about 9.47, about 13.26, about 15.73, about 18.31 and about 22.41 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by PXRD diffractogram peaks at about 9.47, about 12.20, about 13.26, about 15.73, about 18.31, and about 22.41 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 characterized by a PXRD diffractogram substantially similar to FIG. 1.

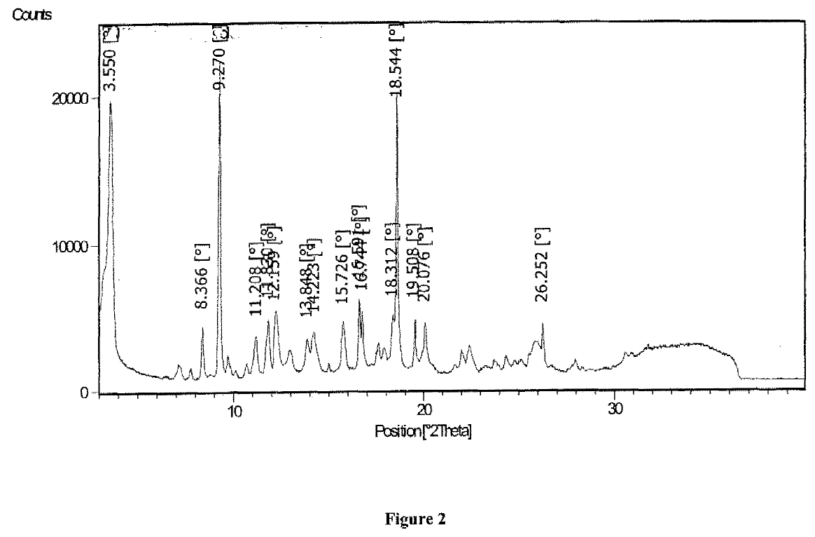

In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by a PXRD diffractogram peak at about 3.55 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by a PXRD diffractogram peak at about 9.27 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by a PXRD diffractogram peak at about 8.37 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by PXRD diffractogram peaks at about 3.55 and about 8.37 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by PXRD diffractogram peaks at about 3.55 and about 9.27 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by PXRD diffractogram peaks at about 8.37 and about 18.54 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by PXRD diffractogram peaks at about 3.55, about 8.37, and about 9.27 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by PXRD diffractogram peaks at about 3.55, about 9.27, and about 18.54 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by PXRD diffractogram peaks at about 8.37, about 12.16, and about 18.54 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by PXRD diffractogram peaks at about 3.55, about 8.37, about 9.27, about 11.21, and about 18.54 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by PXRD diffractogram peaks at about 3.55, about 8.37, about 9.27, about 11.21, about 16.60, and about 18.54 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 characterized by a PXRD diffractogram substantially similar to FIG. 2.

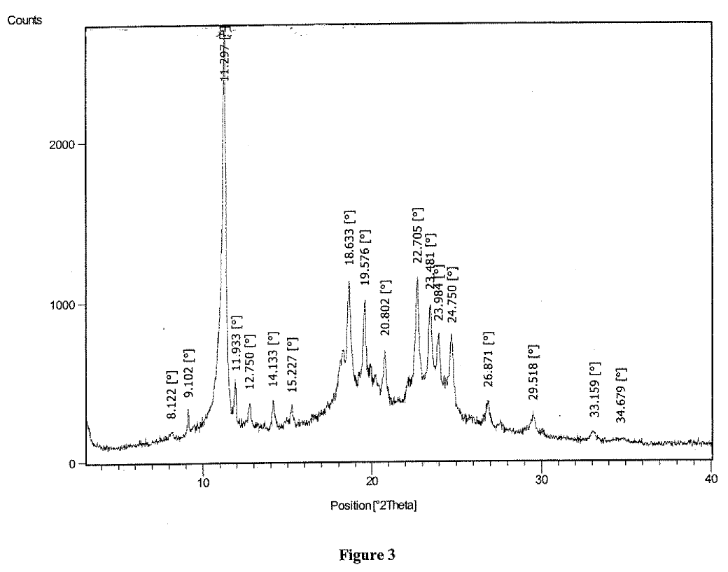

In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by a PXRD diffractogram peak at about 11.30 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by a PXRD diffractogram peak at about 18.63 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by a PXRD diffractogram peak at about 22.71 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by PXRD diffractogram peaks at about 11.30 and about 18.63 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by PXRD diffractogram peaks at about 22.71 and about 23.48 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by PXRD diffractogram peaks at about 8.12 and about 9.10 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by PXRD diffractogram peaks at about 11.30, about 18.63, and about 22.71 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by PXRD diffractogram peaks at about 11.30, about 19.58, and about 22.71 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by PXRD diffractogram peaks at about 9.10, about 11.30, and about 20.80 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by PXRD diffractogram peaks at about 9.10, about 11.30, about 18.63, about 19.58, and about 22.71 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by PXRD diffractogram peaks at about 9.10, about 11.30, about 20.80, about 23.48, and about 24.75 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 characterized by a PXRD diffractogram substantially similar to FIG. 3.

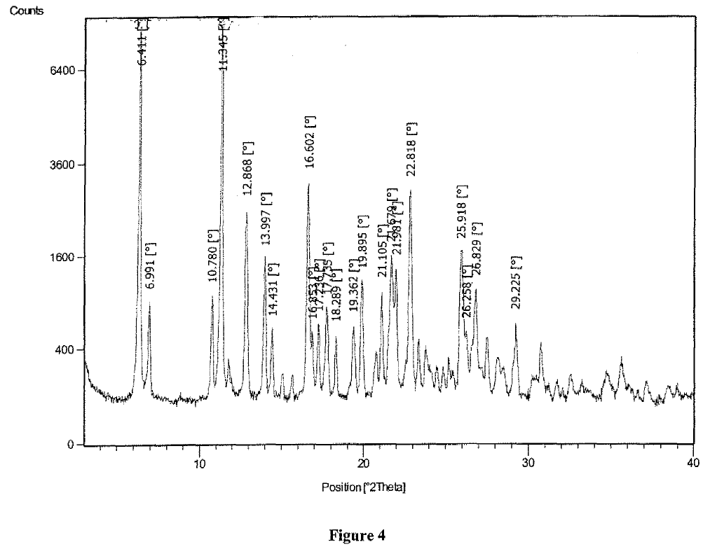

In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by a PXRD diffractogram peak at about 6.41 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by a PXRD diffractogram peak at about 6.99 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by a PXRD diffractogram peak at about 11.35 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by PXRD diffractogram peaks at about 6.99 and about 11.35 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by PXRD diffractogram peaks at about 6.41 and about 11.35 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by PXRD diffractogram peaks at about 10.78 and about 12.87 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by PXRD diffractogram peaks at about 6.41, about 6.99, and about 11.35 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by PXRD diffractogram peaks at about 11.35, about 12.87, and about 16.60 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by PXRD diffractogram peaks at about 6.41, about 11.35, and about 16.60 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by PXRD diffractogram peaks at about 6.41, about 6.99, about 11.35, about 12.87, and about 16.60 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by PXRD diffractogram peaks at about 6.41, about 6.99, about 11.35, about 12.87, about 14.00, about 16.60, and about 19.90 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 characterized by a PXRD diffractogram substantially similar to FIG. 4.

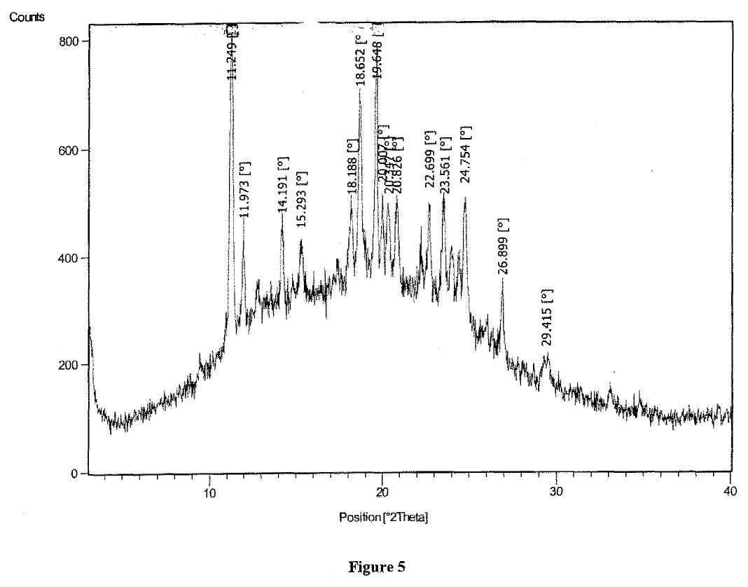

In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by a PXRD diffractogram peak at about 11.25 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by a PXRD diffractogram peak at about 11.97 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by a PXRD diffractogram peak at about 19.65 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by PXRD diffractogram peaks at about 11.25 and about 11.97 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by PXRD diffractogram peaks at about 11.25 and about 19.65 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by PXRD diffractogram peaks at about 11.97 and about 19.65 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by PXRD diffractogram peaks at about 11.25, about 11.97, and about 19.65 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by PXRD diffractogram peaks at about 11.25, about 11.97, and about 20.01 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by PXRD diffractogram peaks at about 11.25, about 20.01, and about 23.56 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by PXRD diffractogram peaks at about 11.25, about 11.97, about 19.65, about 20.01, and about 23.56 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by PXRD diffractogram peaks at about 11.25, about 11.97, about 14.19, about 19.65, about 20.01, about 22.70, and about 23.56 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 characterized by a PXRD diffractogram substantially similar to FIG. 5.

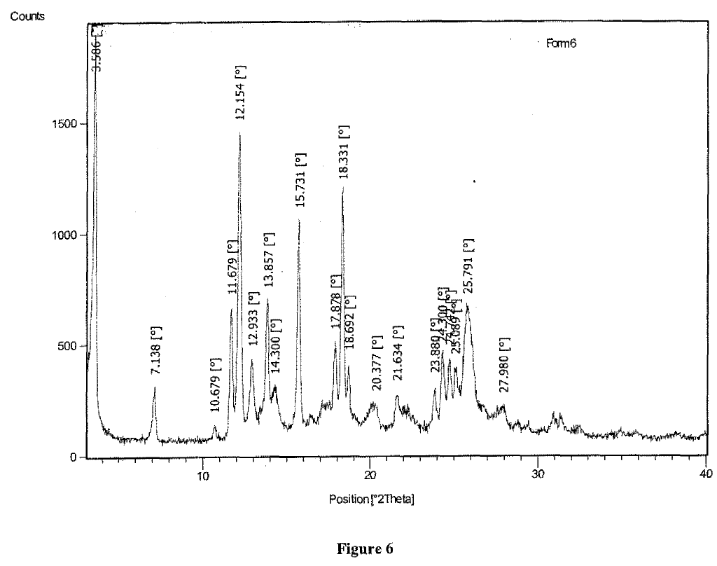

In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by a PXRD diffractogram peak at about 7.14 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by a PXRD diffractogram peak at about 12.93 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by a PXRD diffractogram peak at about 21.63 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by PXRD diffractogram peaks at about 7.14 and about 12.93 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by PXRD diffractogram peaks at about 7.14 and about 21.63 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by PXRD diffractogram peaks at about 12.93 and about 21.63 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by PXRD diffractogram peaks at about 7.14, about 12.93, and about 21.63 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by PXRD diffractogram peaks at about 7.14, about 12.93, and about 23.88 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by PXRD diffractogram peaks at about 10.68, about 12.93, and about 21.63 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by PXRD diffractogram peaks at about 7.14, about 10.68, about 12.93, about 14.30, and about 21.63 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by PXRD diffractogram peaks at about 7.14, about 10.68, about 12.15, about 12.93, about 14.30, about 15.73, and about 21.63 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 characterized by a PXRD diffractogram substantially similar to FIG. 6.

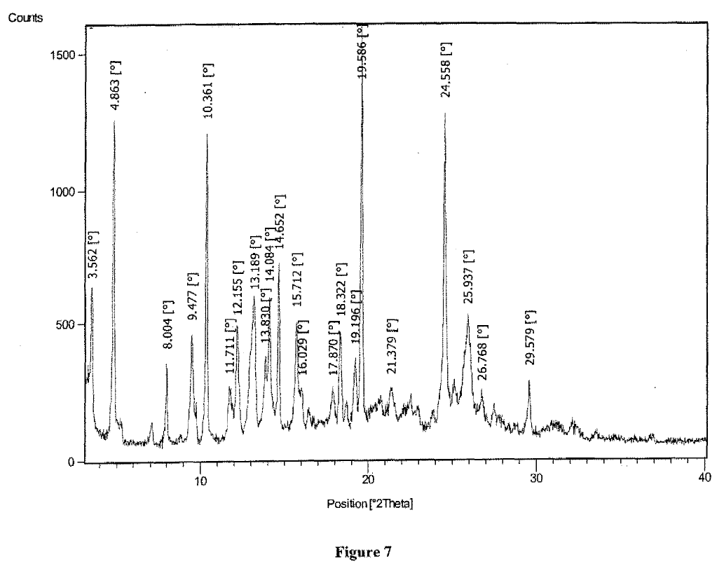

In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by a PXRD diffractogram peak at about 4.86 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by a PXRD diffractogram peak at about 10.36 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by a PXRD diffractogram peak at about 8.00 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by PXRD diffractogram peaks at about 4.86 and about 8.00 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by PXRD diffractogram peaks at about 10.36 and about 19.59 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by PXRD diffractogram peaks at about 4.86 and about 10.36 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by PXRD diffractogram peaks at about 4.86, about 8.00, and about 9.48 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by PXRD diffractogram peaks at about 10.36, about 14.65, and about 19.59 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by PXRD diffractogram peaks at about 4.86, about 12.16, and about 13.19 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by PXRD diffractogram peaks at about 4.86, about 8.00, about 9.48, about 10.36, and about 19.59 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by PXRD diffractogram peaks at about 4.86, about 8.00, about 9.48, about 10.36, about 13.19, about 14.65, and about 19.59 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 characterized by a PXRD diffractogram substantially similar to FIG. 7.

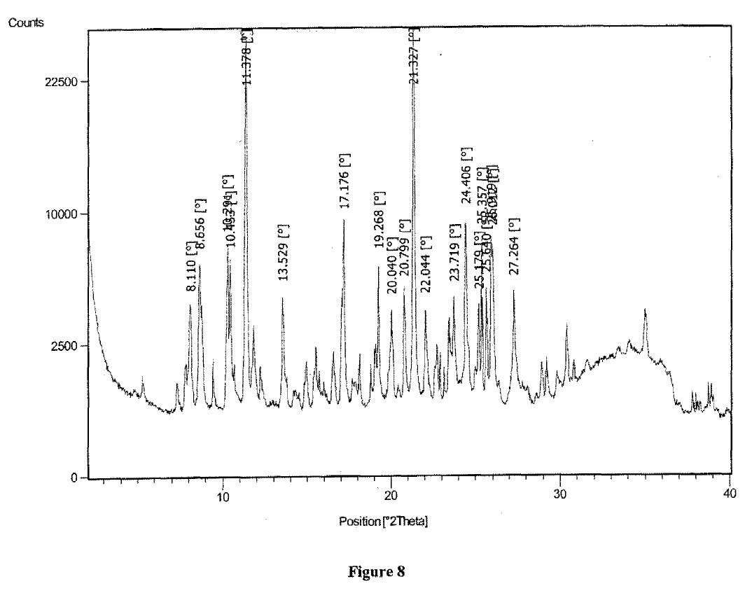

In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by a PXRD diffractogram peak at about 8.11 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by a PXRD diffractogram peak at about 11.38 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by a PXRD diffractogram peak at about 13.53 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by PXRD diffractogram peaks at about 8.11 and about 8.66 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by PXRD diffractogram peaks at about 11.38 and about 13.53 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by PXRD diffractogram peaks at about 8.11 and about 11.38 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by PXRD diffractogram peaks at about 8.11, about 8.66, and about 11.38 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by PXRD diffractogram peaks at about 8.11, about 13.53, and about 17.18 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by PXRD diffractogram peaks at about 8.66, about 11.38, and about 13.53 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by PXRD diffractogram peaks at about 8.11, about 8.66, about 11.38, about 13.53, and about 17.18 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by PXRD diffractogram peaks at about 8.11, about 8.66, about 11.38, about 13.53, about 17.18, about 19.27, and about 21.33 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 characterized by a PXRD diffractogram substantially similar to FIG. 8.

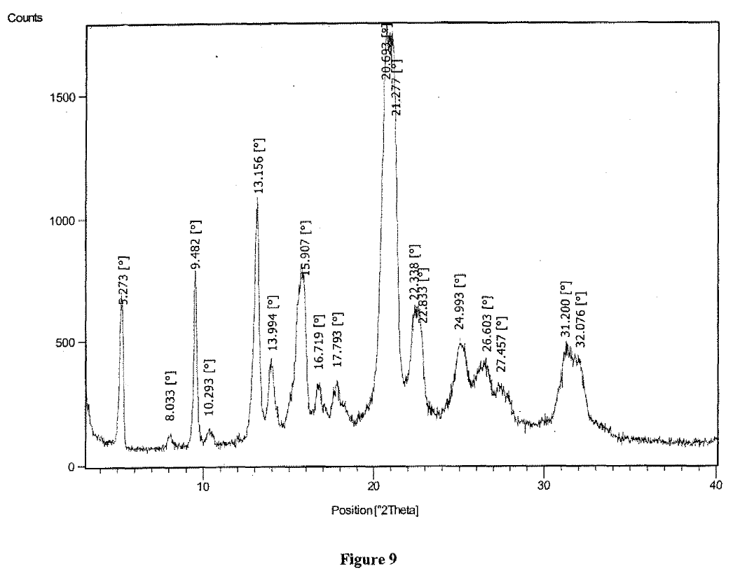

In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by a PXRD diffractogram peak at about 5.27 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by a PXRD diffractogram peak at about 9.48 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by a PXRD diffractogram peak at about 13.16 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by PXRD diffractogram peaks at about 5.27 and about 9.48 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by PXRD diffractogram peaks at about 13.16 and about 13.99 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by PXRD diffractogram peaks at about 5.27 and about 13.16 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by PXRD diffractogram peaks at about 5.27, about 9.48, and about 13.16 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by PXRD diffractogram peaks at about 5.27, about 13.16, and about 13.99 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by PXRD diffractogram peaks at about 5.27, about 9.48, and about 13.99 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by PXRD diffractogram peaks at about 5.27, about 8.03, about 9.48, about 13.16, and about 13.99 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by PXRD diffractogram peaks at about 5.27, about 8.03, about 9.48, about 10.29, about 13.16, about 13.99, and about 16.72 degrees 2-theta. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 characterized by a PXRD diffractogram substantially similar to FIG. 9.

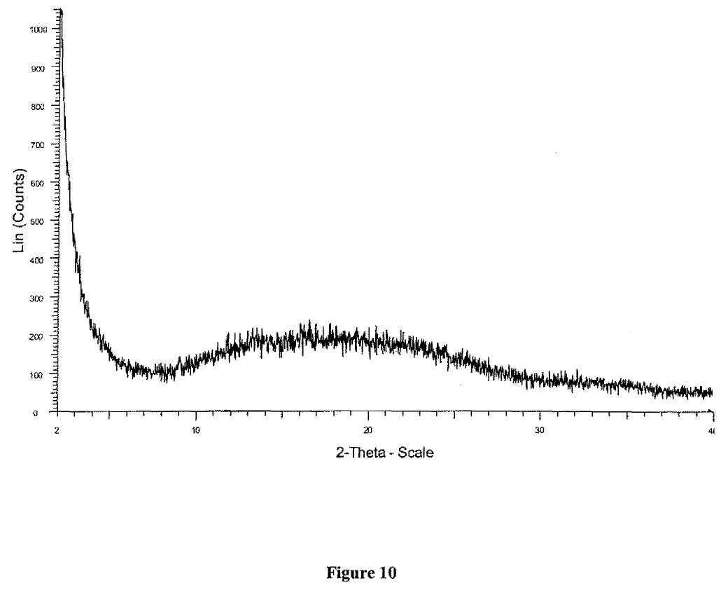

In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid in an amorphous form. In another embodiment, the present invention comprises (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid in an amorphous form characterized by a PXRD diffractogram substantially similar to FIG. 10.

In another embodiment, the present invention comprises a polymorph of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, and methods of making and using the same. In another embodiment, the present invention comprises a solvate or a hydrate of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, and methods of making and using the same. In another embodiment, the present invention comprises a polymorph of a hydrate or a solvate of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, and methods of making and using the same. In another embodiment, the present invention comprises a co-crystal of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, and methods of making and using the same. In another embodiment, the present invention comprises an amorphous form of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, and methods of making and using the same.

In another embodiment, the present invention provides a method of making a polymorph of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, comprising:
(a) providing (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid and a solvent;
(b) contacting said (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid with said solvent; and
(c) evaporating said solvent to form a solid.

In another embodiment, said solvent is an aqueous or an organic solvent, such as, water, hexane, methanol, ethyl acetate, nitromethane, ethanol, acetonitrile, acetone, dichloromethane, isopropyl alcohol, butanol, toluene, or 1,4-dioxane. In a specific embodiment, said solvent is selected from the group consisting of: water, hexane, ethyl acetate, ethanol, acetonitrile, acetone, dichloromethane, isopropyl alcohol, butanol, and toluene. In another embodiment, said solvent is a mixture of two or more solvents.

In another embodiment, the method of making a polymorph of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid further comprises heating said solid to promote complete evaporation of solvent.

In another embodiment, the present invention provides a method of making a solvate of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, comprising:
(a) providing (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid and a solvent;
(b) contacting said (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid with said solvent; and
(c) evaporating said solvent to form a solid.

In another embodiment, said solvent is an aqueous or an organic solvent, such as, water, hexane, methanol, ethyl acetate, nitromethane, ethanol, acetonitrile, acetone, dichloromethane, isopropyl alcohol, butanol, toluene, or 1,4-dioxane. In a specific embodiment, said solvent is selected from the group consisting of: methanol, ethyl acetate, nitromethane, acetonitrile, dichloromethane, and toluene. In another embodiment, said solvent is a mixture of two or more solvents.

In another embodiment, the present invention provides a method of making an amorphous form of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, comprising:
(a) providing (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid and a solvent;
(b) contacting said (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid with said solvent; and
(c) evaporating said solvent to form a solid.

In another embodiment, said solvent is an aqueous or an organic solvent, such as, water, hexane, methanol, ethyl acetate, nitromethane, ethanol, acetonitrile, acetone, dichloromethane, isopropyl alcohol, butanol, toluene, or 1,4-dioxane. In a specific embodiment, said solvent is 1,4-dioxane. In another embodiment, said solvent is a mixture of two or more solvents.

The thermodynamically most stable polymorph (form 6) can be crystallized from acetone, butanol, ethanol, and isopropyl alcohol. The six solvates identified were obtained from acetonitrile, ethyl acetate, dichloromethane, methanol, nitromethane, and toluene. It was observed by Thermogravimetric Analyzer (TGA) that the solvents were evaporated when the solvates melted. An amorphous form was observed from the sample precipitated from dioxane.

In another embodiment of the present invention, a method of treating a mammal or preventing a mammal from suffering from increased vascular resistance or cardiac insufficiency is provided, comprising administering to said mammal an effective amount of a (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid polymorph, solvate, or amorphous form. In another embodiment of the present invention, a method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries is provided, comprising administering to said mammal an effective amount of a (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid polymorph, solvate, or amorphous form. In another embodiment, said mammal is a human.

In another embodiment, the present invention includes the preparation of a medicament comprising a (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid polymorph, solvate, or amorphous form. Such a medicament can be used for treating or preventing inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, in a mammal in need of such treatment. In another embodiment, said mammal is a human.

Pharmaceutical dosage forms of a (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid polymorph, solvate, or amorphous form can be administered in several ways including, but not limited to, oral administration. Oral pharmaceutical compositions and dosage forms are exemplary dosage forms. Optionally, the oral dosage form is a solid dosage form, such as a tablet, a caplet, a hard gelatin capsule, a starch capsule, a hydroxypropyl methylcellulose (HPMC) capsule, or a soft elastic gelatin capsule. Liquid dosage forms may also be provided by the present invention, including such non-limiting examples as a suspension, a solution, syrup, or an emulsion.

A (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid solid form can be administered by controlled- or delayed-release means. Controlled-release pharmaceutical products generally have a common goal of improving drug therapy over that achieved by their non-controlled release counterparts. Ideally, the use of an optimally designed controlled-release preparation in medical treatment is characterized by a minimum of Active Pharmaceutical Ingredient (API) substance being employed to cure or control the condition in a minimum amount of time. Advantages of controlled-release formulations generally include: 1) extended activity of the API; 2) reduced dosage frequency; 3) increased patient compliance; 4) usage of less total API; 5) reduction in local or systemic side effects; 6) minimization of API accumulation; 7) reduction in blood level fluctuations; 8) improvement in efficacy of treatment; 9) reduction of potentiation or loss of API activity; and 10) improvement in speed of control of diseases or conditions. (Kim, Cherng-ju, Controlled Release Dosage Form Design, 2 Technomic Publishing, Lancaster, Pa.: 2000).

Like the amounts and types of excipients, the amounts and specific type of active ingredient in a dosage form may differ depending on factors such as, but not limited to, the route by which it is to be administered to mammals. However, typical dosage forms of the invention comprise a (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′ -carboxylic acid solid form, in an amount of from about 0.10 mg to about 1.00 g, from about 0.2 mg to about 500.0 mg, or from about 1.0 mg to about 250.0 mg. Non-limiting examples include 0.2 mg, 0.50 mg, 0.75 mg, 1.0 mg, 1.2 mg, 1.5 mg, 2.0 mg, 3.0 mg, 5.0 mg, 7.0 mg, 10.0 mg, 25.0 mg, 50.0 mg, 100.0 mg, 250.0 mg, and 500.0 mg dosages. In a particular embodiment, the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form for use in such a composition is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6. The dosage amounts described herein are expressed in amounts of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid and do not include the weight of any water or solvent molecules.

The dosages, however, may be varied depending upon the requirement of the patients, the severity of the condition being treated and the compound being employed. The use of either daily administration or post-periodic dosing may be employed.

The dosage amounts can be administered in single or divided doses. In other embodiments, the present invention is directed to compositions comprising (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid forms as described herein and one or more diluents, carriers, and/or excipients suitable for the administration to a mammal for the treatment or prevention of one or more of the conditions described herein.

The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid forms of the present invention may also be used to prepare pharmaceutical dosage forms other than the oral dosage forms described above, such as topical dosage forms, parenteral dosage forms, transdermal dosage forms, and mucosal dosage forms. For example, such forms include creams, lotions, solutions, suspensions, emulsions, ointments, powders, patches, suppositories, and the like.

The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid forms of the present invention can be characterized by the TGA or DSC data, or by any one, any two, any three, any four, any five, any six, any seven, any eight, any nine, any ten, or any single integer number of PXRD 2-theta angle peaks, or by any combination of the data acquired from the analytical techniques described above.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

### Methods

### Differential Scanning Calorimetry

DSC analysis of each sample was performed using a Q100 Differential Scanning Calorimeter (TA Instruments, New Castle, Del., U.S.A.), which uses Thermal Advantage™ version 4.1.0 for operating instrument. In addition, the analysis software used was Universal Analysis 2000 for Windows 2000/XP, version 4.1D; Build 4.1.0.16 (Copyright © 1998-2004 TA Instruments-Water LLC).

For all of the DSC analyses, an aliquot of a sample was weighed into either a standard aluminium pan (Pan part #900786.091; lid part #900779.901) or a hermetic aluminium pan (Pan part #900793.901; lid part #900794.901 (TA Instruments, New Castle Del. USA)). Non-solvated samples were loaded into standard pans and were sealed either by crimping for dry samples or press fitting for wet samples (such as slurries). Solvated samples (including hydrates) were loaded into hermetic pans and hermetically sealed. The sample pan was loaded into the Q100 Differential Scanning Calorimeter, which is equipped with an autosampler, and a thermogram was obtained by individually heating the same using the control software at a rate of 10° C./minute from Tₘᵢₙ (typically 25° C.) to Tₘₐₓ (typically 275° C.) using an empty aluminium pan as a reference. Dry nitrogen (compressed nitrogen, grade 4.8 (BOC Gases, Murray Hill, N.J. USA)) was used as a sample purge gas and was set at a flow rate of 50 mL/minute. Thermal transitions were viewed and analyzed using the analysis software provided with the instrument.

### Thermogravimetric Analysis

Thermogravimetric analysis (TGA) of samples was performed using a Q50 Thermogravimetric Analyzer (TA Instruments, New Castle, Del., U.S.A.), which uses Thermal Advantage™ version 4.1.0 for operating instrument. In addition, the analysis software used was Universal Analysis 2000 for Windows 2000/XP, version 4.1D; Build 4.1.0.16 (Copyright © 1998-2004 TA Instruments-Water LLC).

For the TGA experiments, the purge gas used was dry nitrogen, the balance purge was 10 mL/minute N₂, and the sample purge was 90 mL/minute N₂.

TGA was performed on the sample by placing a sample in a platinum pan. The starting temperature was typically 25° C. with a heating rate of 10 degrees C./minute, and the ending temperature was 275° C.

### Powder X-Ray Diffraction

Powder x-ray diffraction patters were obtained using PANalytical (formerly Philips Analytical) X'Pert PRO X-ray diffraction system equipped with the X'Celerator detector. All samples were analyzed as received. The samples were either back loaded into conventional XRD holders or placed on zero background holder. Using the X-Celerator, all samples were scanned from 3 to 40° 2θ at a step size of 0.0165° 2θ and a time per step of 10.16 seconds. The effective scan speed was 0.2067° 2θ/s. Instrument voltage and current settings of 45 kV and 40 mA were employed (detailed parameters are listed in the table below).

XRD Hardware

| Instrument | Manufacturer | Model # | Serial # |
|---|---|---|---|
| Diffractometer | Philips | X'PERT PRO MPD | DY1410 |
| Personal | Gateway | ATXSTF FED PRO | 0024749373 |
| Computer | | M1000 | |
| Monitor | Gateway | VX920 | M105049937 |
| Printer | H-P | Desk Jet 990 | MX1311S15M |

XRD Software

Philips X-Pert Data Collector Software, Version 2.0
Philips X'Pert High Score Software, Version 1.0b

Sample Spinner platform (PW3064/00) was mainly used in this work, which is also routinely set up for characterization of drug substances. It is designed to rotate samples fitted in PW 18xx sample holders about their axis. The purpose of spinning is to bring more crystallites into the diffraction position in order to reduce the influence of particle statistics on the measurements. Two types of sample holders, including zero background holder (ZBH, PW1817/32) and cavity sample holder (CSH, PW 1811/16) was used, which has also been set up for route measurements in the laboratory to obtain quality data with minimum amount of materials. The ZBH is made from single crystal silicon, with dimensions of 32 diameter and 2 mm thickness. It is used together with circular sample holder or ring (PW1813/32). ZBH can be used to mount very small amounts of powder (<1 mg), glass capillary, and fibers. The CSH, assembled with a common bottom plate (PW1811/00) and ring, is designed for the manual or semi-automatic preparation of powder samples that can be back-loaded or front-loaded. The bottom plate supports the powder and enables loading into the PW3064/00 Sample Spinner. The diameter of the cavity to be filled is 16 mm. The ring is 2.4 mm thick. A couple of hundreds milligram powder of drug compound is required to fill the CSH. Both ZBH and CSH holders were run on sample changer (PW3065/01), which is used to automatically load and unload samples onto a sample stage and is set up to run batches of routine measurements. The sample changer utilizes removable magazine containing 15 sample positions. The sample arm loads the sample from the magazine onto the sample spinner. The data collection for all these samples was completed in three batches and took only several hours.

### Procedures:

Crystalline powders were gently ground with pestle or spatula when particles are too large. About 10 mg of sample was placed on ZBH holder and a thin layer of the sample was made either using a powder press block or piston (PW 1770/10, powder sample preparation kit) with little extra force or any kind of block with flat surfaces. A strong mechanic force can results in the decrease in crystallinity or polymorphs. In general, a sample was first scanned, as is, from 3 to 50°. Then, the sample was mixed thoroughly with about 10% of standard reference material (SRM 675) and re-scanned at same conditions. It is not necessary for the mixture to be packed as thin as for the sample. Both the sample and its mixture with SRM 675 can also loaded into sample magazine at same time to run batch provided the amount of sample is sufficient.

Raw data was processed using the application software of X'Pert HighScore. The background of a raw data was first determined automatically (Sonneveld and Viser, 1975), and then peak search was performed using the minimum 2^{nd} derivative approach. The peak positions of the sample mixture with internal standard SRM 675 were corrected from the known reflection of d₀₀₁ at 2θ=9.98104. After adjusted, some isolated distinguish peaks near that region diffracted from the drug compound were then chosen as references to rectify the peak positions of the X-ray powder pattern from the pure sample. Therefore, the overlap of peaks between sample and internal standard is avoided in this study.

### EXAMPLES

### Example 1

### (4R)-1,2,3,5-TETRAHYDRO-SPIRO[4H-1-BENZAZEPINE-4,1′-[2]CYLOPENTENE]-3′-CARBOXYLIC ACID

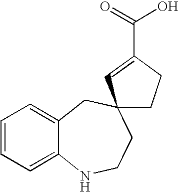

In a 3-necked, 5-L, round-bottomed flask fitted with an air-pump stirrer, (4R)-2,3,4,5-tetrahydrobenzazepine-4-spiro-3′-cyclopent-1′-ene-carboxylic acid-(1R,4S)-7,7-dimethyl-2-oxo-bicyclo[2.2.1]heptane-methanesulfonate (500 g, 1.05 mol) was suspended in H₂O (2 L) to yield a reaction mixture with a pH of about 3-4. With an addition funnel, saturated aqueous NaHCO₃ solution was added slowly to the mixture until pH 6. CH₂Cl₂ (1 L) was then added and the slurry mixture stirred for 1 h. Any remaining starting material in the mixture was then filtered off. The layers were separated and the aqueous layer extracted with CH₂Cl₂ (2×150 mL). The combined organic layer was dried with Na₂SO₄, filtered and concentrated to yield (4R)-1,2,3,5-tetrahydro-spiro[4H-1-benzazepine-4,1′-[2]cylopentene]-3′-carboxylic acid as a dark gray solid.

To the remaining starting material, the process was repeated again until all the salts were completely converted to free acid.

All of crude (4R)-1,2,3,5-tetrahydro-spiro[4H-1-benzazepine-4,1′-[2]cylopentene]-3′-carboxylic acid was combined, suspended in EtOAc/hexanes (1:1) stirring overnight at room temperature and then filtered to yield (4R)-1,2,3,5-tetrahydro-spiro[4H-1-benzazepine-4,1′-[2]cylopentene]-3′-carboxylic acid as a gray solid in 88% yield.

MS (electro spray, positive mode), (M+H)⁺ 244.1.0.

¹H NMR (400 MHz, CDCl₃) δ: 7.09-7.01 (m, 2H), 6.76 (t, J=6.3 Hz, 1H), 6.77 (s, 1H), 6.72 (d, J=7.6 Hz, 1H), 3.17-3.14 (m, 1H), 3.07-3.05 (m, 1H), 2.82 (dd, J=53.3, 13.64 Hz, 2H), 2.71-2.54 (m, 2H), 1.92-1.68 (m, 4H).

### Example 2

### (4R)-1,2,3,5-TETRAHYDRO-SPIRO[4H-1-BENZAZEPINE-4,1′-[2]CYLOPENTENE]-3′-CARBOXYLIC ACID ETHYL ESTER

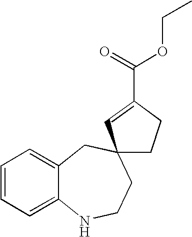

In a 3-necked, 3-L, round-bottomed flask fitted with an inlet thermometer and air-pump stirrer, (4R)-1,2,3,5-tetrahydro-spiro[4H-1-benzazepine-4,1′-[2]cylopentene]-3′-carboxylic acid (225.0 g, 0.92 mol) was slurried in EtOH (1 L). The flask was chilled in an ice bath and slowly, conc. H₂SO₄ (90 g) was added while maintaining the internal temperature between 15 and 25° C. The ice bath was removed after the addition was complete and the reaction was stirred overnight at room temperature. The reaction was 98% complete after the reaction mixture was heated for another 5 days at 40° C. The reaction mixture was concentrated to a black oil, diluted in CH₂Cl₂ (1 L), then washed with H₂O (2×500 mL), saturated NaHCO₃ solution (1×1 L) and saturated NaCl solution (1×1 L). The extracted organic layer was dried with Na₂SO₄, filtered and concentrated to yield (4R)-1,2,3,5-tetrahydro-spiro[4H-1-benzazepine-4,1′-[2]cylopentene]-3′-carboxylic acid ethyl ester as a black oil. Crude (4R)-1,2,3,5-tetrahydro-spiro[4H-1-benzazepine-4,1′-[2]cylopentene]-3′-carboxylic acid ethyl ester was purified by filtration chromatography (silica gel column: 14 cm OD, 8 cm in height and eluting with 4/1 hexanes/EtOAc). The desired fractions were combined to recover (4R)-1,2,3,5-tetrahydro-spiro[4H-1-benzazepine-4,1′-[2]cylopentene]-3′-carboxylic acid ethyl ester as dark red oil. Filtration chromatography was repeated again and fractions containing the product were combined to yield (4R)-1,2,3,5-tetrahydro-spiro[4H-1-benzazepine-4,1′-[2]cylopentene]-3′-carboxylic acid ethyl ester as a yellow oil.

MS (electro spray, positive mode), (M+H)⁺ 272.1.

¹H NMR (400 MHz, CDCl₃) δ: 7.08-7.01 (m, 2H), 6.83 (t, J=7.3 Hz, 11H), 6.71 (d, J=7.8 Hz, 1H), 6.63 (t, J=2.0 Hz, 1H), 4.18 (dd, J=14.4, 7.3 Hz, 2H), 3.77 (br s, 1H), 3.19-3.13 (m, 1H), 3.07-3.0 (m, 1H), 2.81 (dd, J=56.6, 13.6 Hz, 2H), 2.70-2.53 (m, 2H), 1.91-1.65 (m, 4H), 1.29 (t, J=7.1 Hz, 3H).

### Example 3

### (4R)-1-[4-(2-CHLORO-5-FLUOROBENZOYL)AMINO-3-METHOXYBENZOYL]-1,2,3,5-TETRAHYDRO{4H-1-BENZAZEPINE-4,1′-[2CYCLOPENTENE1-3′-CARBOXYLIC ACID ETHYL ESTER

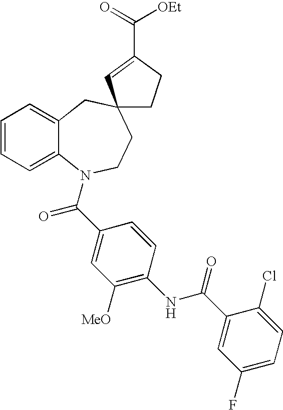

In a dried 11-neck, 3-L, round-bottomed flask fitted with an air-pump stirrer, combined ester (4R)-1,2,3,5-tetrahydro {4H-1-benzazepine-4,1′-[2]cyclopentene]-3′-carboxylic acid ethyl ester (105 g, 0.39 mol) and 4-(2-chloro-5-fluoro-benzoyl)amino-3-methoxy-benzoyl chloride (146 g, 0.43 mol) in CH₂Cl₂ (1 L). The reaction mixture (suspension) was chilled using an ice bath to 0° C. and triethylamine (65 mL, 0.47 mol, 1.2 eq) was added slowly during a period of 15 minutes. The ice bath was removed and reaction mixture allowed to warm up-to room temperature. After 30 minutes HPLC analysis indicated the reaction was complete. The reaction mixture was quenched with H₂O (500 mL) and the layers separated. The organic layer was washed with saturated NaHCO₃ solution (1×500 mL) and saturated NaCl solution (1×500 mL). The extracted organic layer was dried with Na₂SO₄ and filtered. The filtrate containing crude product was concentrated to oil and purified by filtration chromatography (silica gel column: 14 cm OD, 8 cm in height and eluting with 4/1 EtOAc/hexanes). The desired fractions were combined yield (4R)-1-[4-(2-chloro-5-fluorobenzoyl)amino-3-methoxybenzoyl]-1,2,3,5-tetrahydro {4H-1-benzazepine-4,1′-[2]cyclopentene]-3′-carboxylic acid ethyl ester as an orange oil.

MS (electro spray, negative mode), (M+H)⁺ 577.0.

¹H NMR (400 MHz, CDCl₃) δ: 8.66 (s, 1H), 8.26 (d, J=8.3 Hz, 1H), 7.48 (dd, J=8.6, 3.0, 1H), 7.41 (dd, J=8.6, 4.5 Hz, 1H), 7.22-7.09 (m, 3H), 7.0 (t, J=7.0 Hz, 1H), 6.94 (s, 1H), 6.75-6.67 (m, 2H), 4.84 (bd, J=48 Hz, 1H), 4.25-4.14 (m, 2H), 3.72 (s, 3H), 3.33 (dd, J=13.4, 4.5 Hz, 1H), 3.16-2.96 (m, 1H), 2.75-2.61 (m, 3H), 2.13-1.93 (m, 2H), 1.79-1.72 (m, 3H), 1.34-1.22 (m, 3H).

### Example 4

### (4R)-1-[4-(2-CHLORO-5-FLUOROBENZOYL)AMINO-3-METHOXYBENZOYL]-1,2,3,5-TETRAHYDRO-SPIRO[4H-1-BENZAZEPINE-4,1′-[2]CYCLOPENTENE]-3′-CARBOXYLIC ACID

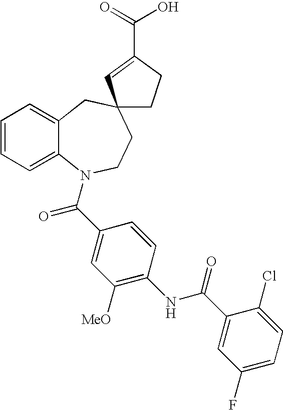

In a 1-necked, 2-L, round-bottomed flask fitted with a magnetic stir bar, (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid ethyl ester (220.0 g, 0.38 mol) was diluted in EtOH/THF (350 mL/350 mL). A hot (ca. 60-70° C.) solution of LiOH (13.7 g, 0.57 mol) in H₂O (200 mL) was slowly added drop-wise to solution over a period of 15 minutes. The reaction mixture was stirred and allowed to cool to room temperature overnight. The reaction mixture was concentrated to an oil, treated with H₂O (1 L), transferred to a separatory funnel and washed with EtOAc (1×500 mL). The aqueous layer was acidified to pH 1-2 using 3 M HCl then extracted with EtOAc (2×500 mL). The extracted organic layer was dried with Na₂SO₄, filtered and concentrated under reduced pressure until precipitation developed in the flask. The precipitated solids were treated with Et₂O/hexanes (600 mL/200 mL) and stirred for 2 h and then filtered. The filtered solids were dried in a high vacuum pump overnight in a rotovap at 60° C. to yield the title compound (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid as a white solid.

mp 178-180° C.

MS (electro spray, negative mode), (M⁺+Na) 571.0

¹H NMR (400 MHz, CDCl₃) δ: 8.66 (s, 1H), 8.26 (d, J=8.3 Hz, 1H), 7.48 (dd, J=8.6, 3.3 Hz, 1H), 7.41 (dd, J=8.8, 4.8 Hz, 1H), 7.23-7.1 (m, 3H), 7.0 (t, J=7.8 Hz, 1H), 6.73-6.67 (m, 2H), 4.86 (bd, J=49.7 Hz, 1H), 3.73 (s, 3H), 3.35 (dd, J=13.6, 5.0 Hz, 1H), 3.15-2.96 (m, 1H), 2.76-2.62 (m, 3H), 2.15-2.0 (m, 2H), 1.82-1.54 (m, 2H)

### Example 5

### SOLID FORMS OF (4R)-1-[4-(2-CHLORO-5-FLUOROBENZOYL)AMINO-3-METHOXYBENZOYL]-1,2,3,5-TETRAHYDRO-SPIRO[4H-1-BENZAZEPINE-4,1′-[2]CYCLOPENTENE]-3′-CARBOXYLIC ACID

### Materials

Compound of Formula (I): (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid,

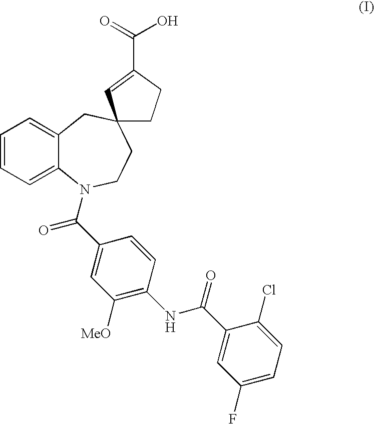

### Crystallization

About 20 mg of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid was transferred into a 4 ml vial. Solvent was added in the vial to make the solution or suspension depending on solubility at about 40° C. on hot plate. The vial was removed from hot plate and kept at room temperature. The caps were put on without sealing. All the vials were placed in the hood for slow evaporation. After the solvent evaporated, the solid was investigated using PXRD, DSC, TGA, and microscope.

### Crystallization Solvents List and Observations

| Solvents | Observation | Observation |
|---|---|---|
| Water | Poor wetability | Suspension |
| Hexane | Poor solubility | Suspension |
| Methanol | Medium solubility | Solution |
| Ethyl acetate | Good solubility | Solution |
| Nitromethane | Good solubility | Solution |
| Ethanol | Medium solubility | Solution |
| Acetonitrile | Good solubility | Solution |
| Acetone | Good solubility | Solution |
| Dichloromethane | Good solubility | Solution |
| Isopropyl alcohol | Medium solubility | Solution |
| Butanol | Medium solubility | Solution |
| Toluene | Solution is not clear | Precipitation |
| 1,4-Dioxane | Good solubility | Solution |

### X-Ray Analysis

The physical state of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid crystallization samples were evaluated using a powder X-ray diffractometer (Philips X'PERT PRO) with X'Celerator detector. The detector is equipped with a real time multiple strip X-ray detection technology such that a high quality powder diffractogram is obtained in only a few minutes of time. The sample was transferred onto a zero background XRD-holder, gently ground and scanned from 2° to 40° 2θ at a scan rate of 0.0167° 2θ/second.

### Results

At least 10 forms were discovered based on distinguished PXRD patterns (all conversions occurred via DSC; such conversions can also occur under ambient conditions at a slower pace).

| Solvent | Form | |
|---|---|---|
| Water | 6 | (polymorph) |
| Hexane | 6 | (polymorph) |
| Methanol | 4 | (solvate converted to an amorphous form) |
| Ethyl acetate | 8 | (solvate converted to form 1) |
| Nitromethane | 9 | (solvate) |
| Ethanol | 6 | (polymorph) |
| Acetonitrile | 7 | (solvate converted to form 1) |
| Acetone | 6 | (polymorph) |
| Dichloromethane | 3 | (solvate converted to form 5) |
| Isopropyl alcohol | 6 | (polymorph) |
| Butanol | 6 | (polymorph) |
| Toluene | 2 | (solvate converted to form 6) |
| 1,4-Dioxane | 10 | (amorphous form) |

### Form 1 (Polymorph)

Form 1 was first observed during DSC (Differential Scanning Calorimetry) analysis of the samples crystallized from acetonitrile (form 7, solvate) and ethyl acetate (form 8, solvate). Both forms 7 and 8 converted to form 1 upon the solvate desolvation. Form 1 has a melting peak at about 185° C. and a heat of fusion of about 60 J/g. The TGA thermograms showed that there were no weight losses in the temperature range near the melting point of form 1, indicating that form 1 is an unsolvated form. Form 1 was determined to be a polymorph, free of solvent and water molecules within the crystal structure. The polymorph exhibited very little weight loss, during TGA analysis, prior to decomposition.

The PXRD (powder X-ray diffraction) pattern of form 1 was obtained using the sample isolated by heating form 7 to 130° C. on DSC and then cooling down to room temperature. The peak positions listed below were confirmed with internal standard. Form 1 can be characterized by any one, any two, any three, any four, any five, or any six or more of the peaks in FIG. 1 including, but not limited to, 3.56, 5.28, 7.08, 7.99, 9.47, 10.65, 11.72, 12.20, 13.26, 13.86, 14.26, 15.73, 17.88, 18.31, 18.67, 20.66, 21.77, 22.41, 24.32, and 25.06 degrees 2-theta. FIG. 1 shows form 1 as converted from form 7.

### Form 2 (Toluene Solvate)

The sample crystallized from toluene was named form 2. The peak positions shown below were confirmed with internal standard. TGA showed that the desolvation of form 2 occurred at about 130° C. Form 2 can be characterized by any one, any two, any three, any four, any five, or any six or more of the peaks in FIG. 2 including, but not limited to, 3.55, 8.37, 9.27, 11.21, 11.83, 12.16, 13.85, 14.22, 15.73, 16.59, 16.74, 18.31, 18.54, 19.51, 20.08, and 26.25 degrees 2-theta.

### Form 3 (Dichloromethane Solvate)

Form 3 was crystallized from dichloromethane and is a solvate. It desolvated at the melting peak ˜104° C. and simultaneously converted to form 5 on DSC (this form 5 subsequently melted with the peak at ˜168° C.). TGA study of form 3 showed ˜0.9% weight loss and desolvation below 150° C., and it became a metastable polymorph, free of solvent and water molecules within the crystal structure. The peak positions shown below were confirmed with internal standard. Form 3 can be characterized by any one, any two, any three, any four, any five, or any six or more of the peaks in FIG. 3 including, but not limited to, 8.12, 9.10, 11.30, 11.93, 12.75, 14.13, 15.23, 18.63, 19.58, 20.80, 22.71, 23.48, 23.98, 24.75, 26.87, 29.52, and 33.16 degrees 2-theta.

### Form 4 (Methanol Solvate)

The sample of this form was crystallized from methanol, showing strong crystallinity. TGA study showed ˜5% weight loss, and its desolvation occurred at 130° C. The peak positions shown below were confirmed with internal standard. Form 4 can be characterized by any one, any two, any three, any four, any five, or any six or more of the peaks in FIG. 4 including, but not limited to, 6.41, 6.99, 10.78, 11.35, 12.87, 14.00, 14.43, 16.60, 17.74, 19.36, 19.90, 21.11, 21.68, 22.82, 25.92, 26.83, and 29.23 degrees 2-theta.

### Form 5 (Polymorph)

Form 5 was converted from the dichloromethane solvate (form 3) upon heating and was a desolvate based on form 3 TGA (Thermogravimetric Analyzer) results. The form 5 material was collected by heating form 3 to 130° C. and cooling down to room temperature. While the two PXRD patterns of forms 3 and 5 are similar, significant differences exist which validate the characterization of two distinct forms. Form 5 melted at about 168° C. with a heat of fusion of about 36 J/g. TGA study of form 5 showed very little weight loss. Its peak positions shown below were confirmed with internal standard. Form 5 can be characterized by any one, any two, any three, any four, any five, or any six or more of the peaks in FIG. 5 including, but not limited to, 11.25, 11.97, 14.19, 15.29, 18.19, 18.65, 19.65, 20.01, 20.35, 20.83, 22.70, 23.56, 24.75, 26.90, and 29.42 degrees 2-theta.

### Form 6 (Polymorph)

Form 6 was crystallized from acetone, butanol, ethanol, isopropyl alcohol, hexane, and water. It melted with a peak at about 203-204° C. and a heat of fusion of about 75-80 J/g. Form 6 has the highest melting temperature and the heat of fusion, indicating it was the thermodynamically most stable polymorph. This result has been confirmed by water slurry study. After an equal amount of forms 1, 5, and 6 were mixed in water for more than 76 hours, forms 1 and 5 converted to form 6. TGA study showed no weight loss.

Its PXRD (degrees 2-theta) peak positions shown below were confirmed with internal standard. Form 6 can be characterized by any one, any two, any three, any four, any five, or any six or more of the peaks in FIG. 6 including, but not limited to, 3.59, 7.14, 10.68, 11.68, 12.15, 12.93, 13.86, 14.30, 15.73, 17.88, 18.33, 18.69, 20.38, 21.63, 23.88, 24.30, 24.74, 25.09, 25.79, and 27.98 degrees 2-theta.

### Form 7 (Acetonitrile Solvate)

Form 7 is an acetonitrile solvate as discussed in the section above describing form 1. This solvate desolvated at 120° C. and converted to form 1. TGA study showed ˜1% weight loss, and desolvation occurred at 123° C. Upon desolvation, it converted to form 1, and ultimately to form 6. The peak positions of form 7 were confirmed with internal standard. Form 7 can be characterized by any one, any two, any three, any four, any five, or any six or more of the peaks in FIG. 7 including, but not limited to, 3.56, 4.86, 8.00, 9.48, 10.36, 11.71, 12.16, 13.19, 14.08, 14.65, 15.71, 18.32, 19.59, 24.56, 25.94, and 29.58 degrees 2-theta.

### Form 8 (Ethyl Acetate Solvate)

Form 8 is an ethyl acetate solvate which melted with a peak at ˜130° C. and converted to form 1 upon desolvation. TGA study showed ˜10% weight loss. The peak positions shown below were confirmed with internal standard. Form 8 can be characterized by any one, any two, any three, any four, any five, or any six or more of the peaks in FIG. 8 including, but not limited to, 8.11, 8.66, 10.29, 10.45, 11.38, 13.53, 17.18, 19.27, 21.33, 24.41, and 27.26 degrees 2-theta.

### Form 9 (Nitromethane Solvate)

Form 9 was crystallized from nitromethane. The PXRD pattern of form 9 was confirmed with internal standard. In addition, TGA study showed very little weight loss. It desolvated around 187° C.

Form 9 can be characterized by any one, any two, any three, any four, any five, or any six or more of the peaks in FIG. 9 including, but not limited to, 5.27, 8.03, 9.48, 10.29, 13.16, 13.99, 15.91, 16.72, 17.79, 20.69, 21.28, 22.34, 24.99, 26.60, and 31.20 degrees 2-theta.

### Form 10 (Amorphous Form)

An amorphous form was observed from the sample precipitated from 1,4-dioxane. The PXRD diffractogram of form 10 is shown in FIG. 10.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1.

2. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 of claim 1, wherein said form 1 is characterized by a powder X-ray diffraction pattern comprising peaks expressed in terms of 2-theta angles, and further wherein said X-ray diffraction pattern comprises a peak at about 9.47 degrees.

3. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 of claim 1, wherein said form 1 is characterized by a powder X-ray diffraction pattern comprising peaks expressed in terms of 2-theta angles, and further wherein said X-ray diffraction pattern comprises a peak at about 13.26 degrees.

4. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 of claim 1, wherein said form 1 is characterized by a powder X-ray diffraction pattern comprising peaks expressed in terms of 2-theta angles, and further wherein said X-ray diffraction pattern comprises peaks at about 9.47 and about 13.26 degrees.

5. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 of claim 1, wherein said form 1 is characterized by a powder X-ray diffraction pattern comprising peaks expressed in terms of 2-theta angles, and further wherein said X-ray diffraction pattern comprises peaks at about 20.66 and about 22.41 degrees.

6. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 of claim 1, wherein said form 1 is characterized by a powder X-ray diffraction pattern comprising peaks expressed in terms of 2-theta angles, and further wherein said X-ray diffraction pattern comprises peaks at about 9.47, about 13.26, and about 20.66 degrees.

7. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 of claim 1, wherein said form 1 is characterized by a powder X-ray diffraction pattern comprising peaks expressed in terms of 2-theta angles, and further wherein said X-ray diffraction pattern comprises peaks at about 9.47, about 13.26, about 20.66, and about 22.41 degrees.

8. (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2.

9. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 of claim 8, wherein said form 2 is characterized by a powder X-ray diffraction pattern comprising peaks expressed in terms of 2-theta angles, and further wherein said X-ray diffraction pattern comprises a peak at about 8.37 degrees.

10. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 of claim 8, wherein said form 2 is characterized by a powder X-ray diffraction pattern comprising peaks expressed in terms of 2-theta angles, and further wherein said X-ray diffraction pattern comprises a peak at about 9.27 degrees.

11. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 of claim 8, wherein said form 2 is characterized by a powder X-ray diffraction pattern comprising peaks expressed in terms of 2-theta angles, and further wherein said X-ray diffraction pattern comprises peaks at about 8.37 and about 9.27 degrees.

12. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 of claim 8, wherein said form 2 is characterized by a powder X-ray diffraction pattern comprising peaks expressed in terms of 2-theta angles, and further wherein said X-ray diffraction pattern comprises peaks at about 8.37, about 9.27, and about 12.16 degrees.

13. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 of claim 8, wherein said form 2 is characterized by a powder X-ray diffraction pattern comprising peaks expressed in terms of 2-theta angles, and further wherein said X-ray diffraction pattern comprises peaks at about 8.37, about 9.27, about 12.16, and about 18.54 degrees.

14. (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3.

15. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 of claim 14, wherein said form 3 is characterized by a powder X-ray diffraction pattern comprising peaks expressed in terms of 2-theta angles, and further wherein said X-ray diffraction pattern comprises a peak at about 11.30 degrees.

16. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 of claim 14, wherein said form 3 is characterized by a powder X-ray diffraction pattern comprising peaks expressed in terms of 2-theta angles, and further wherein said X-ray diffraction pattern comprises a peak at about 18.63 degrees.

17. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 of claim 14, wherein said form 3 is characterized by a powder X-ray diffraction pattern comprising peaks expressed in terms of 2-theta angles, and further wherein said X-ray diffraction pattern comprises peaks at about 11.30 and about 18.63 degrees.

18. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 of claim 14, wherein said form 3 is characterized by a powder X-ray diffraction pattern comprising peaks expressed in terms of 2-theta angles, and further wherein said X-ray diffraction pattern comprises peaks at about 11.30, about 18.63, and about 19.58 degrees.

19. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 of claim 14, wherein said form 3 is characterized by a powder X-ray diffraction pattern comprising peaks expressed in terms of 2-theta angles, and further wherein said X-ray diffraction pattern comprises peaks at about 11.30, about 18.63, about 19.58, and about 22.71 degrees.

20. (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4.

21. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 of claim 20, wherein said form 4 is characterized by a powder X-ray diffraction pattern comprising peaks expressed in terms of 2-theta angles, and further wherein said X-ray diffraction pattern comprises a peak at about 6.41 degrees.

22. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 of claim 20, wherein said form 4 is characterized by a powder X-ray diffraction pattern comprising peaks expressed in terms of 2-theta angles, and further wherein said X-ray diffraction pattern comprises a peak at about 6.99 degrees.

23. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 of claim 20, wherein said form 4 is characterized by a powder X-ray diffraction pattern comprising peaks expressed in terms of 2-theta angles, and further wherein said X-ray diffraction pattern comprises peaks at about 6.41 and about 6.99 degrees.

24. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 of claim 20, wherein said form 4 is characterized by a powder X-ray diffraction pattern comprising peaks expressed in terms of 2-theta angles, and further wherein said X-ray diffraction pattern comprises peaks at about 6.41, about 6.99, and about 11.35 degrees.

25. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 of claim 20, wherein said form 4 is characterized by a powder X-ray diffraction pattern comprising peaks expressed in terms of 2-theta angles, and further wherein said X-ray diffraction pattern comprises peaks at about 6.41, about 6.99, about 11.35, and about 12.87 degrees.

26. (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5.

27. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 of claim 26, wherein said form 5 is characterized by a powder X-ray diffraction pattern comprising peaks expressed in terms of 2-theta angles, and further wherein said X-ray diffraction pattern comprises a peak at about 11.25 degrees.

28. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 of claim 26, wherein said form 5 is characterized by a powder X-ray diffraction pattern comprising peaks expressed in terms of 2-theta angles, and further wherein said X-ray diffraction pattern comprises a peak at about 11.97 degrees.

29. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 of claim 26, wherein said form 5 is characterized by a powder X-ray diffraction pattern comprising peaks expressed in terms of 2-theta angles, and further wherein said X-ray diffraction pattern comprises a peak at about 19.65 degrees.

30. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 of claim 26, wherein said form 5 is characterized by a powder X-ray diffraction pattern comprising peaks expressed in terms of 2-theta angles, and further wherein said X-ray diffraction pattern comprises peaks at about 11.25 and about 11.97 degrees.

31. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 of claim 26, wherein said form 5 is characterized by a powder X-ray diffraction pattern comprising peaks expressed in terms of 2-theta angles, and further wherein said X-ray diffraction pattern comprises peaks at about 19.65 and about 23.56 degrees.

32. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 of claim 26, wherein said form 5 is characterized by a powder X-ray diffraction pattern comprising peaks expressed in terms of 2-theta angles, and further wherein said X-ray diffraction pattern comprises peaks at about 11.25, about 11.97, and about 19.65 degrees.

33. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 of claim 26, wherein said form 5 is characterized by a powder X-ray diffraction pattern comprising peaks expressed in terms of 2-theta angles, and further wherein said X-ray diffraction pattern comprises peaks at about 11.25, about 11.97, about 19.65, and about 23.56 degrees.

34. (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6.

35. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 of claim 34, wherein said form 6 is characterized by a powder X-ray diffraction pattern comprising peaks expressed in terms of 2-theta angles, and further wherein said X-ray diffraction pattern comprises a peak at about 7.14 degrees.

36. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 of claim 34, wherein said form 6 is characterized by a powder X-ray diffraction pattern comprising peaks expressed in terms of 2-theta angles, and further wherein said X-ray diffraction pattern comprises a peak at about 12.93 degrees.

37. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 of claim 34, wherein said form 6 is characterized by a powder X-ray diffraction pattern comprising peaks expressed in terms of 2-theta angles, and further wherein said X-ray diffraction pattern comprises a peak at about 21.63 degrees.

38. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 of claim 34, wherein said form 6 is characterized by a powder X-ray diffraction pattern comprising peaks expressed in terms of 2-theta angles, and further wherein said X-ray diffraction pattern comprises peaks at about 12.93 and about 21.63 degrees.

39. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 of claim 34, wherein said form 6 is characterized by a powder X-ray diffraction pattern comprising peaks expressed in terms of 2-theta angles, and further wherein said X-ray diffraction pattern comprises peaks at about 11.68 and about 12.93 degrees.

40. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 of claim 34, wherein said form 6 is characterized by a powder X-ray diffraction pattern comprising peaks expressed in terms of 2-theta angles, and further wherein said X-ray diffraction pattern comprises peaks at about 12.93, about 15.73 and about 21.63 degrees.

41. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 of claim 34, wherein said form 6 is characterized by a powder X-ray diffraction pattern comprising peaks expressed in terms of 2-theta angles, and further wherein said X-ray diffraction pattern comprises peaks at about 7.14, about 12.93, about 15.73 and about 18.33 degrees.

42. (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7.

43. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 of claim 42, wherein said form 7 is characterized by a powder X-ray diffraction pattern comprising peaks expressed in terms of 2-theta angles, and further wherein said X-ray diffraction pattern comprises a peak at about 4.86 degrees.

44. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 of claim 42, wherein said form 7 is characterized by a powder X-ray diffraction pattern comprising peaks expressed in terms of 2-theta angles, and further wherein said X-ray diffraction pattern comprises a peak at about 10.36 degrees.

45. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 of claim 42, wherein said form 7 is characterized by a powder X-ray diffraction pattern comprising peaks expressed in terms of 2-theta angles, and further wherein said X-ray diffraction pattern comprises peaks at about 4.86 and about 10.36 degrees.

46. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 of claim 42, wherein said form 7 is characterized by a powder X-ray diffraction pattern comprising peaks expressed in terms of 2-theta angles, and further wherein said X-ray diffraction pattern comprises peaks at about 4.86, about 10.36, and about 13.19 degrees.

47. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 of claim 42, wherein said form 7 is characterized by a powder X-ray diffraction pattern comprising peaks expressed in terms of 2-theta angles, and further wherein said X-ray diffraction pattern comprises peaks at about 4.86, about 8.00, about 10.36, and about 13.19 degrees.

48. (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8.

49. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 of claim 48, wherein said form 8 is characterized by a powder X-ray diffraction pattern comprising peaks expressed in terms of 2-theta angles, and further wherein said X-ray diffraction pattern comprises a peak at about 8.11 degrees.

50. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 of claim 48, wherein said form 8 is characterized by a powder X-ray diffraction pattern comprising peaks expressed in terms of 2-theta angles, and further wherein said X-ray diffraction pattern comprises a peak at about 11.38 degrees.

51. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 of claim 48, wherein said form 8 is characterized by a powder X-ray diffraction pattern comprising peaks expressed in terms of 2-theta angles, and further wherein said X-ray diffraction pattern comprises peaks at about 8.11 and about 8.66 degrees.

52. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 of claim 48, wherein said form 8 is characterized by a powder X-ray diffraction pattern comprising peaks expressed in terms of 2-theta angles, and further wherein said X-ray diffraction pattern comprises peaks at about 8.11, about 8.66, and about 11.38 degrees.

53. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 of claim 48, wherein said form 8 is characterized by a powder X-ray diffraction pattern comprising peaks expressed in terms of 2-theta angles, and further wherein said X-ray diffraction pattern comprises peaks at about 8.11, about 8.66, about 11.38, and about 17.18 degrees.

54. (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9.

55. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 of claim 54, wherein said form 9 is characterized by a powder X-ray diffraction pattern comprising peaks expressed in terms of 2-theta angles, and further wherein said X-ray diffraction pattern comprises a peak at about 5.27 degrees.

56. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 of claim 54, wherein said form 9 is characterized by a powder X-ray diffraction pattern comprising peaks expressed in terms of 2-theta angles, and further wherein said X-ray diffraction pattern comprises a peak at about 9.48 degrees.

57. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 of claim 54, wherein said form 9 is characterized by a powder X-ray diffraction pattern comprising peaks expressed in terms of 2-theta angles, and further wherein said X-ray diffraction pattern comprises peaks at about 5.27 and about 9.48 degrees.

58. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 of claim 54, wherein said form 9 is characterized by a powder X-ray diffraction pattern comprising peaks expressed in terms of 2-theta angles, and further wherein said X-ray diffraction pattern comprises peaks at about 5.27, about 9.48, and about 13.16 degrees.

59. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 of claim 54, wherein said form 9 is characterized by a powder X-ray diffraction pattern comprising peaks expressed in terms of 2-theta angles, and further wherein said X-ray diffraction pattern comprises peaks at about 5.27, about 9.48, about 13.16, and about 15.91 degrees.

60. Amorphous (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid.

61. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 of claim 1, wherein said form 1 is characterized by a DSC thermogram, and further wherein said DSC thermogram comprises a melting point at about 185 degrees C.

62. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 of claim 26, wherein said form 5 is characterized by a DSC thermogram, and further wherein said DSC thermogram comprises a melting point at about 168 degrees C.

63. The (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 of claim 34, wherein said form 6 is characterized by a DSC thermogram, and further wherein said DSC thermogram comprises a melting point at about 203-204 degrees C.

64. A method of making a polymorph of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, comprising:
(a) providing (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid and a solvent;
(b) contacting said (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid with said solvent; and
(c) evaporating said solvent to form a solid.

65. The method of claim 64, wherein said solvent is selected from the group consisting of: water, hexane, methanol, ethyl acetate, nitromethane, ethanol, acetonitrile, acetone, dichloromethane, isopropyl alcohol, butanol, toluene, 1,4-dioxane, and any mixture thereof.

66. The method of claim 64, further comprising heating said solid to promote complete evaporation of solvent.

67. The method of claim 64, wherein said formed solid is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1.

68. The method of claim 64, wherein said formed solid is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5.

69. The method of claim 64, wherein said formed solid is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6.

70. A method of making a solvate of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, comprising:
(a) providing (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid and a solvent;
(b) contacting said (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid with said solvent; and
(c) evaporating said solvent to form a solid.

71. The method of claim 70, wherein said formed solid is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2.

72. The method of claim 70, wherein said formed solid is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3.

73. The method of claim 70, wherein said formed solid is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4.

74. The method of claim 70, wherein said formed solid is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7.

75. The method of claim 70, wherein said formed solid is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8.

76. The method of claim 70, wherein said formed solid is (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9.

77. A method of making an amorphous form of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, comprising:
(a) providing (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid and a solvent;
(b) contacting said (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid with said solvent; and
(c) evaporating said solvent to form a solid.

78. A method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, comprising administering to said mammal an effective amount of the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 1 of claim 1.

79. A method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, comprising administering to said mammal an effective amount of the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 2 of claim 8.

80. A method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, comprising administering to said mammal an effective amount of the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 3 of claim 14.

81. A method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, comprising administering to said mammal an effective amount of the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 4 of claim 20.

82. A method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, comprising administering to said mammal an effective amount of the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 5 of claim 26.

83. A method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, comprising administering to said mammal an effective amount of the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 of claim 34.

84. A method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, comprising administering to said mammal an effective amount of the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 7 of claim 42.

85. A method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, comprising administering to said mammal an effective amount of the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 8 of claim 48.

86. A method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, comprising administering to said mammal an effective amount of the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 9 of claim 54.

87. A method of treating a mammal or preventing a mammal from suffering from inner ear disorders, aggression, anxiety, obsessive-compulsive disorders, hypertension, dysmenorrhea, congestive heart failure/cardiac insufficiency, coronary vasospasm, liver cirrhosis, renal vasospasm, renal failure, diabetic nephropathy, hyponatremia, edema, ischemia, stroke, thrombosis, water retention, nephritic syndrome, or central nervous system injuries, comprising administering to said mammal an effective amount of the amorphous (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid of claim 60.

88. A pharmaceutical composition comprising the (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid form 6 of claim 34.
